(19) United States
(12) Patent Application Publication
(10) Pub. No.: US 2006/0275902 A1
(43) Pub. Date: Dec. 7, 2006

(54) Cell and virus catabolism

(75) Inventor: Tia Patrice Griffin, Spokane, WA (US)

Correspondence Address: Tia Patrice Griffin, 1428 E. Glass Avenue, Spokane, WA, 99207, US

(73) Assignee: Tia Patrice Griffin

(21) Appl. No.: 11/360,186

(22) Filed: Feb. 21, 2006

Related U.S. Application Data
(63) Continuation-in-part of application No. 60/655,020, filed on Feb. 22, 2005.

(51) Int. Cl. C12N 5/08

(52) U.S. Cl. 435/381

(57) ABSTRACT

Disease at cellular levels have been overlooked. To date, we can create clones but have no cure to Cancer, Aids, and certain bacteriums. Why haven't we looked a little deeper? Why not break up a cell wall rendering dangerous DNA useless? Why not break through protein coats or jackets of HIV cells to prevent DNA from reproducing itself? Once unprotected, DNA particles cannot live and reproduce themselves in the bloodstream. Creating a medication that has an Immune System Builder+Catabolizer to Destroy HIV, Bacterial, and Viral Cells+Building Up Healthy Bone Marrow is the cure to these diseases.

It is well known knowledge that the cell wall of the normal human cell is responsible for cell shape and protection of the plasma membrane. It is also a given that the human cell is made up of many components. These components include oligosaccharide antigens, cholesterol, protein, and phospholipids.

Since many of these components can be metabolized in the body, I feel we can consider the same individual breakdown for the cell wall. How can this be accomplished? By studying the composition of these components thoroughly. My suggestion is to weaken, break down, and create cell wall lysis. Why am I suggesting this? Simply because when we break down the cell wall of a normal human cell, we can then figure out how to do the same to bacterial cells, viral cells, and cancer cells for the purpose of curing many diseases. My idea is to stop the cell growth of these particular cells mentioned while in their earlier stages. One might say “Well, we already accomplish this through Chemotherapy as it pertains to cancer.” However, in my opinion, Chemotherapy kills all the cells in certain areas of the body. Chemotherapy is a destroyer of the body and is a very debilitating and dangerous procedure that many times leads to death. I have seen cancer patients grow weak because they can barely endure the treatments. It appears to kill so many cells that the body is too weak to rebuild the new necessary cells it needs to create stasis, survival, healing, and strength.

A brief description of the drawings in figures one and two explain that in the normal cell, the oligosaccharide antigens ( a monosaccharide that cannot be decomposed by hydrolysis such as fructose, galactose, or glucose), lie on the outer portion of the cell wall. Underneath the oligosaccharide layer, one finds cholesterol, protein, and phospholipids attached with an extra layer of oligosaccharide antigens lined somewhat before the inside portion of a cell. The oligosaccharide antigens are short chained carbohydrates with 3 to 10 sugar units or 3 to 10 monosaccharides. If these oligosaccharides cannot be broken down by hydrolysis, then another means should be considered. First, we must figure out which oligosaccharides make up the monosaccharide covering of the normal cell wall. Second, we must figure out how to break down this particular complex carbohydrate lining of the cell wall. Once we can break down the cell wall, we can cause plasma membrane rupture. Plasma membrane rupture creates loss of the cytoplasm and eventually cell lysis. Penicillin and many other antibiotics already create this reaction in many cases. However, not every disease is fully cured by penicillin and antibiotics. And, many times we have to hope that supra-infection does not take place if medications are not properly taken as required. If we can obtain the capability to destroy all bacterial, cancerous, and viral cell walls, we will create a cure for many diseases. My catabolism process then continues by that simple breakdown of the cell wall. This can be accomplished by chemical decomposition of the cell walls mentioned. Chemical decomposition causes a substance to split into simpler compounds by the addition and use of water elements (hydrolysis) and debranching enzymes. If we can create enzymes that act similar to human digestive enzymes (which break the bonds that hold glucose molecules together), we can break down and metabolize the human cell wall. This metabolism can cause the protein, cholesterol, and phospholipids portion of the cell wall to change to amino acids. The amino acids can then continue as usual to pass unchanged through the intestinal walls into the blood. These amino acids then travel to the portal vein into the liver and then safely into the general circulation from which they are absorbed by the tissues based on the specific amino acids needed by that tissue to make its own protein. The unmetabolized amino acids can be converted to urea. The carbohydrate portion of the cell wall could be metabolized to maltose which converts to glucose ( used for energy) or (converted to fat) and safely stored as adipose tissues as usual.

The Bacterial Cell Wall

A brief description of the drawing in figure three explain that the bacterial cell wall differs from the human cell. The Bacterial Cell Wall is made up of many intricate components just like the human cell wall. However, the bacterial cell has gram positive and gram negative cell walls. The gram positive cell wall consists of:

Teichoic acids

Lipoteichoic acids and

Peptidoglycan-By metabolizing the peptidoglycan layers we may be able to weaken the bacterial cell walls. The wall of teichoic acids would not have anything to bind to for survival. This could cause the plasma membrane to be exposed which will eventually cause cell lysis.

The gram negative cell wall consists of:

O-polysaccharide

Lipid A-Both O-polysaccharides and lipids can be metabolized and used in the body for energy.

Porin Protein

Phospholipid

Lipoprotein-Both phospholipids and lipoproteins can be broken down as well. These make up the outer membrane of the gram negative bacterial cell wall. Without the outer membrane, the cell wall has no protection.

Enzymes and other active substances

Peptidoglycan—A carbohydrate that can be broken down and metabolized. The connection here is that each of these cell wall structures can be broken down, catabolized, to create cell wall lysis and control of bacterial, viral, and cancer cell synthesis. One other natural way to create cell wall catabolism is by way of an enzyme called lysozyme (enzymes found in phagocytes, neutrophils, macrophages, tears, saliva, sweat, and other body secretions). If my suggestions are correct or even close, our own body secretions may have enzymes that can destroy certain cell walls.

The Cancer Cell

A brief description of the drawing in figure four explains the Neoplasm. When we look at a cancer cell we find that a cancer cell is created when a group of cells form a malignancy and in some cases a tumor. We already have the means of sometimes finding cancer cells in their early stages. Many times, these cells are found prior to their grouping and forming into a malignancy or tumor. However, the good thing is that we are still talking about cells aren't we? Therefore, there can still be a process of catabolism. There can still be an enzyme that surrounds the bad cells, infiltrating, and then destruction of those cells before they form into a tumor.

The Viral Cell

A brief description of the drawings in figures five and six explain the relationship between the viral cell when cell catabolism is implemented. The drawings in figure five relate to the Cell Catabolism Process while the drawing in Figure six visibly describes the need for Protein Coat Lysis to complete this process of destroying the Viral Cell which stop these cells ability to mutate. The Virus is the most interesting portion of my idea of Cell Wall Catabolism and Lysis Process. Many viruses have particular shells on their outer portions. They have an envelope and a capsid that protects them while they create havoc inside what was once a normal cell. This is where we can implement the old idea of getting to know your enemy. When it comes to HIV, for example, I understand that this virus has a certain intelligence. Its cells multiply and reshape so quickly that it has been impossible to control or even slow down. Yet, there still seems to be one thing that might have been overlooked. This one thing is that even though the cell changes shape, the makeup of the cell does not change. This fact gives us the opportunity to fight our enemy by surrounding it and destroying it no matter what the changing shape becomes. Currently, the viral cell penetrates into a normal cell creating multiplication of the virus. This then causes cell destruction and a release of virus particles. My idea process is to look at and consider what the capsid and envelope of the HIV viral cells are made of. If we can break down the protein jacket before it reaches the normal cell by way of the use of enzymes, we can cause protein coat lysis. For example, lysosomes have the power to break down protein coats in the body. When the protein coat is destroyed, it leaves the nucleic acid coat defenseless. Once defenseless, the DNA inside the viral cell is vulnerable. It has no protection and cannot continue to attach itself to a healthy normal cell in the body. It becomes useless and is eventually destroyed.

Useful Medications

A brief description of the drawings in Figure Seven visibly describe the components of medications that need to be included with the specific enzymes previously mentioned while the cells are being destroyed in this process. My process leads me to basic territory of medications that would be useful to cause this destruction of the HIV Viral Cell and hopefully bacterial cells, as well as cancer cells. This process could also in the future, possibly cure shingles, herpes, and other viruses. To implement medications in this process, there needs to be synergistic medications created that treat both the symptom of the diseases mentioned and the affects on the body after treatments have taken place. During treatment, a balance of intake and output is a must. Since damaging viral cells are being destroyed through cell catabolism, newer RBC's should be developing in the body. At the same time, WBC build up is a must to assist the body with repair and protection from infection. These medications must consist of three important items which are:

1. Immune System Builder+Catabolizer to Destroy HIV, Bacterial, and Viral Cells+Building Up Healthy Bone Marrow.

2. WBC Producer+Enzyme that breaks the bond that hold glucose molecules together+RBC Producers-Iron, Cobalt, Copper

3. Essential Amino Acids+Specific Vitamins-Folic Acid and Vitamin B

4. Glutathione—Barleys are immune system builders that create glutathione and have the three amino acids needed for the body during treatment.

5. Pegademase Bovine—

6. (Adenosine Deaminase; ADA)—There are proteins that accumulate to harm wbc's without the assistance of ADA. ADA is needed because it decreases the risk of infection.

7. RM-10 UltraZorbe

A brief description of the drawing in Figure seven and number four explains the purpose and function of the bone marrow which is of utmost importance as it relates to the blood cells within the individual body. EPO stimulates the bone marrow in the medullan cavity which produces more red blood cells. Every 120 days the human body recreates new red blood cells. A balance these three: the immune system builder, Cell catabolism, and RBC production via medications could produce the cure for the various cellular problems mentioned throughout this process.

Safety Balance

The safety measures to consider are the levels of medications administered during treatment. We know that when abnormal RBC's have been destroyed, their iron is released. To prevent the dangers of iron overload or further destruction of glutathione, a medical doctor would need to monitor the levels of the medications given. The medical doctor would also monitor the blood levels of the patient if possible.

### SUMMARY

Disease at cellular levels have been overlooked. To date, we can create clones but have no cure to Cancer, Aids, and certain bacteriums. Why haven't we looked a little deeper? Why not break up a cell wall rendering dangerous DNA useless? Why not break through protein coats or jackets of HIV cells to prevent DNA from reproducing itself? Once unprotected, DNA particles cannot live and reproduce themselves in the bloodstream. Creating a medication that has an Immune System Builder+Catabolizer to Destroy HIV, Bacterial, and Viral Cells+Building Up Healthy Bone Marrow is the cure to these diseases.

### DESCRIPTION OF DRAWINGS

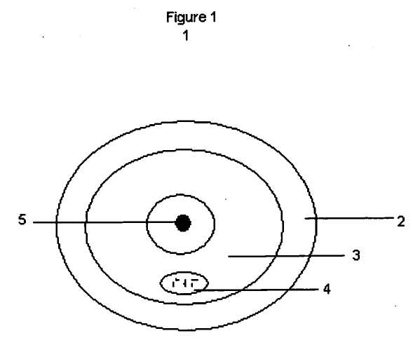

(See FIG. 1).
1. Titled: The Human Cell
2. The Cell Wall
3. The Cytoplasm of a Cell
4. The Ribosomes found within the Cytoplasm
5. The Nucleus which pertains both the DNA and RNA of the Cell

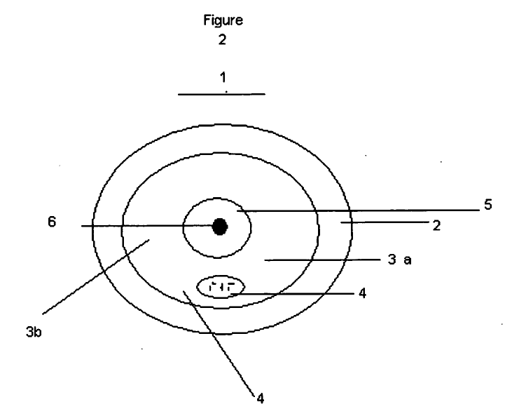

(See FIG. 2)
1. Four Layered View of the Normal Cell
2. The oligosaccharides, Antigens-fructose, galactose, glucose
3a. Protein
   b. Cholesterol
4. Phospholipids
5. More oligosaccharides on both sides
6. Cell Nucleus where we can find the Virus bug for HIV/Aids for example.

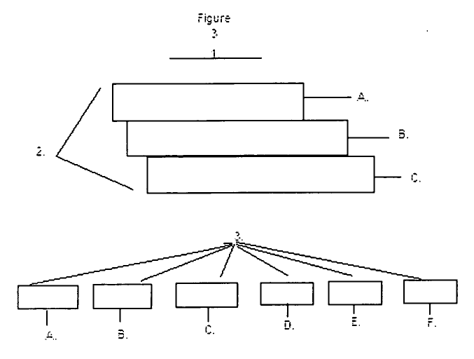

(See FIG. 3)
1. Title: Breaking Down the Bacterial Cell Wall Before it Synthesizes
2. Gram Positive Cell Wall Layers
3. Gram Negative Cell Wall Layers-Both positive and negative cell wall layers can be infiltrated from the outside into destroy the bad cells. If we find a workable enzyme, we will still destroy both the good cells and the bad cells. The difference is that the poison (toxic chemicals) caused from chemotherapy during cancer treatment, for example, will not be a factor. Therefore, the body will be weak but will have the ability to replenish its cells as needed. In my opinion, this would then promote the health and strength needed to assist the body in its attempt to overcome disease instead of the disease overcoming the body.

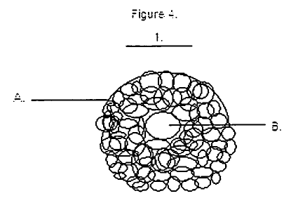

(See FIG. 4)
1. The Neoplasm—Here we can see that the neoplasm is still a formation of tissue. Since tissue is still a group or collection of similar cells, the cells can be destroyed.

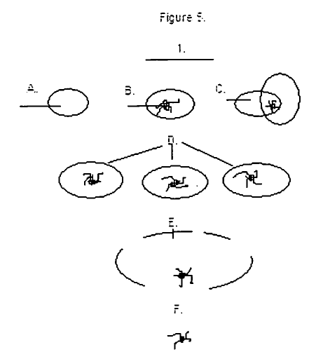

(See FIG. 5)
1. Title: Cell Catabolism Process for the Viral Cell
   A. Normal Cell
   B. Viral Cell
   C. Viral Cell Infiltration
   D. Viral Cell Multiplication
   E. Cell Catabolism and Destruction of Shell Envelope
   F. Outcome—The Viral Bug has no protection. It cannot live or survive without A covering. Therefore, it becomes useless in it's attempts to create new viral Cells.

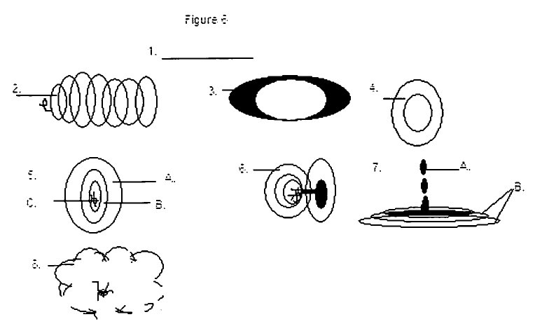

(See FIG. 6)
1. Title: The Virus/Protein Coat Catabolism
2. The Protein Jacket and the Nucleic Acid Coil
3. The Protein Shell
4. Process of Attacking the Normal Cell
   A. The Normal Cell
   B. The Infiltration
5 The Viral Cell Created and Duplication
6 Enzyme and Lysosome (E & L) Solution
   A. (E & L) Infiltration
   B. HIV Cell Rupture

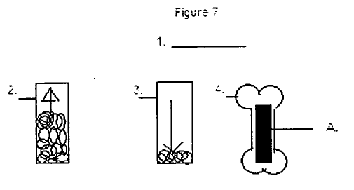

(See FIG. 7)
1. Title: Treatment Goal
2. Build Up of WBC'S (White Blood Cells)—Glutathione—Barleys are immune system builders.
3. Destroy Viral Cells—Using enzymes and lysosomes
4. Rebuild and Replenish RBC'S (Red Blood Cells)—Recreating new white and red blood cells using similar medicines like erythroportin (EPO).

1. My claim is that Cell Catabolism Process can metabolizes the cell wall of a normal cell, the bacterial cell, pre-cancerous cells, and HIV cells by the use of enzymes (lysozymes, and lysosomes) causing cell wall lysis. This lysis causes the destruction of the unwanted cells.

2. I also claim that treatment to cure these diseases and bacterium consists of a created medication containing a balance of Metabolizing enzymes, Immune System Builders, and components that Build Up Healthy Bone Marrow to create the Red Blood Cells (RBC's) needed for health and healing.
